(19) United States
(12) Patent Application Publication
(10) Pub. No.: US 2007/0086952 A1
(43) Pub. Date: Apr. 19, 2007

(54) Rapid Acting and Prolonged Acting Inhalable Insulin Preparations

(75) Inventors: Solomon S. Steiner, Mount Kisco, NY (US); Roderike Pohl, Sherman, CT (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Biodel, Inc.

(21) Appl. No.: 11/537,335

(22) Filed: Sep. 29, 2006

Related U.S. Application Data
(60) Provisional application No. 60/721,698, filed on Sep. 29, 2005.

(51) Int. Cl. A61K 38/28; A61K 9/12

(52) U.S. Cl. 424/45; 514/3; 514/440; 514/54; 514/562; 514/566; 424/195.17; 424/774

(57) ABSTRACT

It has been discovered that by combining a chelator, such as ethylenediaminetetraacetic acid, with an acidifier, such as citric acid, the insulin is absorbed much more rapidly than in the absence of the chelator and acidifier, with a commercially available rapid, intermediate or long lasting insulin such as glargine, one can increase and/or prolong the bioavailability of the insulin mixture. The formulations are suitable for administration by injection or to a mucosal surface such as the pulmonary or oral regions, although subcutaneous injection is preferred.

### CROSS-REFERENCE TO RELATED APPLICATIONS

This application claims priority to U.S. Ser. No. 60/721,608 entitled “Rapid Acting Inhalable Insulin Preparations” filed Sep. 29, 2005, by Solomon S. Steiner.

### BACKGROUND OF THE INVENTION

The present invention is generally in the field of insulin formations, and is specifically formulations of insulin that are more rapid acting and/or have a more prolonged or enhanced period of activity.

When a healthy individual begins a meal, he or she experiences a natural spike of insulin that is released by the pancreas, called the first phase insulin release. Currently available human insulin preparations used by sufferers of diabetes do not replicate the natural first-phase insulin spike. Instead, insulin enters the bloodstream slowly, over a period of several hours. As a consequence, patients with diabetes have inadequate levels of insulin present at the initiation of a meal and have too much insulin in their system between meals. Having too little insulin at the beginning of the meal causes abnormally high blood glucose levels (hyperglycemia) which are the cause of all the long term disabilities associated with diabetes, (such as retinopathies, neuropathies, nephropathies, bed sores and amputations). Because they have too much insulin at the end of the meal, they are prone to a condition known as hypoglycemia, which is an abnormally low level of blood glucose between meals. Hypoglycemia can result in loss of mental acuity, confusion, increased heart rate, hunger, sweating, faintness and, at very low glucose levels, loss of consciousness, coma and even death.

While products that have recently been developed by several major pharmaceutical companies (such as Lilly, Novo-Nordisk, Aventis), at a cost of hundreds of millions of dollars, are a marked improvement over the preceding generation of insulin products, they still are absorbed too slowly to mimic the natural insulin spike.

In fall 2005, Pfizer/Nektar received an FDA advisory committee recommendation to approve their inhalable insulin product. Other inhalable insulin products, such as those of Lilly/ Alkermes and Novo/Aridigm, are in the process of obtaining regulatory approval. Their pharmacokinetics is faster than injectable regular human insulin, but not as fast as the rapid acting insulin analogs, such as HUMALOG®.

These products can be vastly improved in their efficacy if they could be made to act more rapidly and mimic the natural initial insulin spike produced by non-diabetic individuals at the beginning of a meal. This is especially important with these pulmonary insulins as their intended use is to provide meal time insulin demand. Furthermore, if these pulmonary insulin formulations could be made to act fast enough to resemble the natural initial insulin spike produced by non-diabetic individuals at the beginning of a meal, then the liver would be able to respond to this rapid change in insulin level by shutting off the conversion of glycogen to glucose. (The production of glucose by the liver by conversion from glycogen is called hepato-gluconeogenisis.) The irony of the disease is that diabetics, who have insufficient insulin levels to adequately handle their glucose, continue to make glucose in the liver while the concentration of blood glucose is increasing from the natural result of digestion. The liver responds only to the rate of change of insulin level; not the absolute insulin level. By causing the blood insulin levels to rise rapidly and terminate hepato-gluconeogenesis, these improved pulmonary insulin formulations would allow diabetic patients to use less insulin, have sufficient insulin at the beginning of a meal and not suffer an excess of insulin between meals. As a result they would have better glycemic control and reduce the risk of both hyperglycemia and hypoglycemia.

It is therefore an object of the present invention to provide a method and reagents to yield a more rapidly acting insulin.

It is a further object of the present invention to provide a longer acting insulin.

### SUMMARY OF THE INVENTION

It has been discovered that by combining a chelator, such as ethylenediaminetetraacetic acid, with an acidifier, such as citric acid, the insulin is absorbed much more rapidly than in the absence of the chelator and acidifier. By mixing this formulation with commercially available rapid acting insulins, it is possible to alter uptake and pharmacokinetic profiles. It has also been determined that by combining regular or intermediate lasting insulin with a chelator and acidifier, with a long lasting insulin such as glargine, one can increase and/or prolong the bioavailability of the insulin mixture. The formulations are suitable for administration by injection or mucosal delivery (oral, sublingual, buccal, vaginal, rectal, nasal or pulmonary), although subcutaneous injection is preferred. Methods for making pulmonary and solid formulations are described.

The examples demonstrate the enhanced rate of uptake obtained by providing a chelator and acidifying agent with rapid acting and long acting insulins. The examples alo demonstrate that for the first four hours after administration, there is no significant difference between administering a long acting insulin, LANTUS®, and insulin (insulin containing chelator and acidifying agent, VIAJECT™) mixed together or administered separately, however, after the first four hours and up to at least eight hours, there is a very large and significant difference with the mixture of VIAJECT™ and LANTUS® having a much greater effect on lowering blood glucose.

### BRIEF DESCRIPTION OF THE DRAWINGS

FIG. 1 is a graph of a filter study demonstrating the decrease in apparent size of insulin molecules (percent of less than 30,000 mw) in the presence of EDTA and citric acid.

FIG. 2 is a graph of the effect of EDTA and citric acid on apparent permeability of the insulin.

FIG. 3 is a graph of blood glucose levels (mg/dl plasma) over time before (baseline period), during a meal, and after the meal, in minutes, comparing separate and mixed administration of LANTUS® and VIAJECT™.

FIG. 4 is a graph of the blood glucose levels with baseline subtracted from blood glucose area under the curve (BG AUC) comparing separate and mixed administration of LANTUS® and VIAJECT™.

### DETAILED DESCRIPTION OF THE INVENTION

Compositions

A. Drugs To Be Administered

In the preferred embodiment, the active agent is insulin or an analog or derivative thereof. The insulin can be recombinant or purified. In the preferred embodiment, the insulin is recombinant human insulin.

The Initial source of insulin for clinical use in humans was from cow, horse, pig or fish pancreases. Insulin from these sources is effective in humans as it is nearly identical to human insulin (three amino acid difference for bovine insulin, one amino acid difference for porcine). Insulin is a protein which has been very strongly conserved across evolutionary time. Differences in suitability of beef, pork, or fish insulin preparations for particular patients have been primarily the result of preparation purity and of allergic reactions to assorted non-insulin substances remaining in those preparations. Purity has improved more or less steadily since the 1920s, but allergic reactions have continued though slowly reducing in severity. Insulin production from animal pancreases was widespread for decades, but there are very few patients today relying on insulin from these sources.

Recombinant human insulin is available from a number of sources. Human insulin is now manufactured for widespread clinical use using genetic engineering techniques, which significantly reduces impurity reactionp roblems. Eli Lilly marketed the first such insulin, HUMULIN®, in 1982.

The commonly used types of insulin are:
Quick-acting, such as insulin lispro—begins to work within 5 to 15 minutes and is active for 3 to 4 hours.

Short-acting, such as regular insulin—starts working within 30 minutes and is active about 5 to 8 hours.

Intermediate-acting, such as NPH, or lente insulin—starts working in 1 to 3 hours and is active 16 to 24 hours.

Long-acting, such as ultralente insulin—starts working in 4 to 6 hours, and is active 24 to 28 hours, and Insulin glurgine or Insulin detemir—both start working within 1 to 2 hours and continue to be active, without peaks or dips, for about 24 hours.

A mixture of NPH and regular insulin—starts working in 30 minutes and is active 16 to 24 hours. There are several variations with different proportions of the mixed insulins.

The dosage depends on the bioavailability and the disease or disorder to be treated, as well as the individual patient. Insulin is generally included in a dosage range of 12 to 2000 IU per human dose. Thus if the insulin has a bioavailability 5-25%, the actual systemic dose delivered to an individual ranges from 3 to 100 IU. For insulin with only 25% bioavailability, an oral dose of 4,000 IU will deliver a 100 IU systemically available dose. For insulin with a much greater bioavailability, such as a 50% bioavailability, the delivery of a 3 IU systemically available dose requires an oral dose of 6 IU.

Both natural and recombinant insulins are available. Human insulin, HUMALIN® R, U 100, is available as a solution for injection from Eli Lilly and Company, single dose 0.1 IU/kg. Insulin lispro, HUMALOG® R, is available as a solution for injection, from Eli Lilly and Company, single dose 0.1 IU/kg. Insulin glargine, LANTUS® R, U 100, is available from Sanofi-Aventis, as a solution for injection, 0.1 IU/kg.

VIAJECT™, insulin solution including EDTA and citric acid, for injection 25 IU/ml prepared from recombinant human insulin, is available from Biodel Inc. (pending FDA approval).

This technology is also useful with parathyroid hormone amino acids, 1-34, PTH, and analogs and derivatives thereof.

Solubilizing Agents

In the preferred embodiment, one or more solubilizing agents are included with the active agent to promote rapid dissolution in aqueous media. Suitable acids include acetic acid, ascorbic acid, citric acid, and hydrochloric acid. For example, if the active agent is insulin, a preferred solubilizing agent is citric acid. Results are best using citric acid as the solubilizer, although ascorbic acid and acetic acid also yield substantial enhancement, while HCl and sulfuric acid yield poor enhancement.

Other suitable solubilizing agents include wetting agents such as polysorbates and poloxamers, non-ionic and ionic surfactants, food acids and bases (e.g. sodium bicarbonate), and alcohols, and buffer salts for pH control.

C. Chelators

In the preferred embodiment, a metal chelator is mixed with the active agent or in a coating surrounding the active agent. The chelator may be ionic or non-ionic. Suitable chelators include ethylenediaminetetraacetic acid (EDTA), citric acid, dimercaptrol (BAL), penicillamine, alginic acid, chlorella, cilantro, alpha lipoic acid, dimercaptosuccinic acid (DMSA), dimercaptopropane sulfonate (DMPS), and oxalic acid. In the preferred embodiment, the chelator is EDTA. The chelator hydrogen bonds with the active agent, thereby masking the charge of the active agent and facilitating transmembrane transport of the active agent. For example, when the active agent is insulin, in addition to charge masking, it is believed that the chelator pulls the zinc away from the insulin, thereby favoring the monomeric form of the insulin over the hexameric form and facilitating absorption of the insulin by the tissues surrounding the site of administration (e.g. mucosa, or fatty tissue). Optionally, the chelator and solubilizing agent are the same compound.

Ions may be part of the active agent, added to the stabilizing agent, mixed with the chelator, and/or included in the coating. Representative ions include zinc, calcium, iron, manganese, magnesium, aluminum, cobalt, copper, or any di-valent metal or transitional metal ion. Zn⁺² has a stronger binding preference for EDTA than Ca⁺².

D. Diluents

Diluents will typically be saline physiological buffered saline, Ringer's or sterile water. The diluent may contain the chelating agent and/or the solubilizing agent.

Preferred ingredients are those that are Generally Regarded As Safe (GRAS) by the US FDA.

II. Methods of Manufacture and Administration

The drugs can be prepared as powders or spray dried particles, and further formulated for administration by injection, pulmonary, or oral or sublingual routes of administration. Pharmaceutical compositions may be formulated in a conventional manner using one or more physiologically acceptable carriers comprising excipients and auxiliaries which facilitate processing of the active compounds into preparations which can be used pharmaceutically. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Proper formulation is dependent upon the route of administration chosen.

The composition may be in the form of a dry powder containing the pharmaceutically active agent and one or more excipient(s). The active agents and excipients may be in the form of particles having the same or different sizes. In one embodiment, the excipient particles are larger than the particles of agent. This will allow the small particles of agent to coat the larger particle so that both particles are administered simultaneously. Typically, the average particle diameter for the agent particles is less than or equal to one-tenth of the average particle diameter for the excipient particles. For sublingual delivery, the large particles generally have diameters greater than 8 μm, preferably greater than 20 μm. The average diameters for the large particles typically range from 8 μm to 500 μm, preferably from 50 μm to 150 μm. The small particles generally have a diameter ranging from 1 nm to 9 μm, preferably from 100 nm to 400 nm. For buccal and nasal administration, the particles generally have similar size ranges to those described from sublingual administration. For pulmonary administration, the large particles typically have an average diameter ranging from 1 μm to 10 μm, preferably from 2 μm to 5 μm; and the small particles typically have an average diameter ranging from 10 nm to 1 μm.

If the particles of excipient have generally the same size, the average diameters will generally be greater than 8 μm, preferably greater than 20 μm, with typical size ranges from 8 μm to 500 μm, and preferably from 50 μm to 150 μm (for sublingual, buccal and nasal administration); and from 1 μm to 10 μm, preferably from 2 μm to 5 μm (for pulmonary administration).

Optionally, the particles are oppositely charged, so that the excipient particles contain one charge and the agent particles contain the opposite charge so that the particles are administered simultaneously. The particles may be charged by blowing them into a chamber formed of plastic surfaces, which impart charge to the particles. Two oppositely charged chambers may be used. The charged particles may be formed by using an acidic solution to make one of the particles, and a basic solution to form the other particles. Alternatively, charge can be transferred through ion discharge (e.g. using a staticizer or destaticizer). If the particles of agent and excipient are oppositely charged, they may have the same average diameter or different average diameters.

In one embodiment, the particles are formed by spraying a solution of drug through an atomizer, into a dryer which removes the solvent, then the particles are further dried in a lyophilizer. In another embodiment, the particles are formed by spraying a solution of drug into liquid nitrogen, which instantly freezes the drug, the particles are then removed and dried. Drug powders can also be prepared using standard drug milling techniques.

A. Injection

In the most preferred embodiment, the formulation is in a form suitable for subcutaneous injection. For injection, the formulations are preferably administered subcutaneously as a liquid. In this embodiment, the formulation is formed by mixing a powdered agent with a liquid diluent that contains a pharmaceutically acceptable liquid carrier and one or more solubilizing agents. In the preferred embodiment, the active agent is insulin, and the diluent contains saline, EDTA and citric acid. Prior to administration the powder and diluent are mixed together to form an injectable composition. In the most preferred embodiment, the insulin is provided as a dry powder and the chelating agent and acidifying agent are provided as a sterile aqueous liquid in an amount suitable for dissolution of the dry powdered insulin. In a typical formulation, the insulin is reconstituted to a dosage concentration of 0.1 IU/kg.

Pulmonary Delivery

In a preferred embodiment for preparation of a pulmonary formulation, an aqueous solution containing one part recombinant human (“rH”) insulin, two parts of a suitable chelating agent such as EDTA, two parts of a suitable acid such as citric acid, 5 parts of a suitable sugar and a small amount of a suitable surfactant is gently and thoroughly mixed to form a clear solution. The solution is sterile filtered through a 0.2 micron filter into a sterile, enclosed vessel. Under sterile conditions, the solution is passed through an appropriately small orifice to make droplets between 0.1 and 10 microns. The solution can be forced under pressure through a nozzle with very small and uniform holes or sprayed out through an ultrasonic nebulizer into a large volume of liquid nitrogen or some other suitable cryogenic liquid. The frozen liquid is then lyophilized to form a uniform dry powder for use in any of a number of dry powder inhalers. The combination of ingredients containing one part insulin, two parts of a suitable chelating agent such as EDTA, two parts of a suitable acid such as citric acid, with or without a small amount of a suitable surfactant, can be used to speed the rate of absorption and bioavailability of an insulin formulation, especially for pulmonary administration. The combination of ingredients containing one part peptide or protein, two parts of a suitable chelating agent such as EDTA, two parts of a suitable acid such as citric acid, and with or without a small amount of a suitable surfactant can also be used to speed the rate of absorption and bioavailability.

Preferred particle or powder sizes are between 1 and 3 microns, although smaller sizes may be used, from nanometers to 2 microns, or larger sizes, from three to five microns, if the particles are porous or otherwise very light.

The particles may be administered using any of a number of different applicators. Suitable methods for manufacture and administration are described in the following U.S. Pat. Nos. 6,592,904; 6,518,239; 6,423,344; 6,294,204; 6,051,256 and 5,997,848 to Inhale (now Nektar); and U.S. Pat. No. 5,985,309; RE37,053; U.S. Pat. Nos. 6,436,443; 6,447,753; 6,503,480; and 6,635,283, to Edwards, et al. (MIT, AIR).

C. Mucosal Delivery

The mixtures may also be formulated for mucosal delivery, such as oral, nasal, buccal, vaginal, rectal or sublingual delivery. Suitable dosage forms include powders, films, wafers, lozenges, capsules, and tablets. In one preferred embodiment, the formulation is a sublingual solid formulation that contains an active agent, and at least one solubilizing agent, along with other standard excipients, such as poly(vinyl alcohol), glycerin, carboxymethol cellulose (CMC), and optionally poly(ethylene glycol) and water. The sublingual composition may be in the form of a dry powder, monolayer, bilayer, or trilayer film, a lyophilized wafer, lozenge, capsule, or a tablet. In addition to the excipients discussed above, these formulations may include one or more of the following.
Diluents and Fillers

Diluents also referred to herein as fillers, are typically necessary to increase the bulk of a solid dosage form so that a practical size is provided for compression of tablets or formation of beads and granules. Suitable fillers include, but are not limited to, dicalcium phosphate dihydrate, calcium sulfate, lactose, sucrose, mannitol, sorbitol, cellulose, microcrystalline cellulose, powdered cellulose, kaolin, sodium chloride, dry starch, hydrolyzed starches, pregelatinized starch, silicone dioxide, titanium oxide, magnesium aluminum silicate, calcium carbonate, compressible sugar, sugar spheres, powdered (confectioner's) sugar, dextrates, dextrin, dextrose, dibasic calcium phosphate dehydrate, glyceryl palmitostearate, magnesium carbonate, magnesium oxide, maltodextrin, polymethacrylates, potassium chloride, talc, and tribasic calcium phosphate.
Binders

Binders are used to impart cohesive qualities to a solid dosage formulation, and thus ensure that a tablet, bead or granule remains intact after the formation of the dosage forms. Suitable binder materials include, but are not limited to, starch, pregelatinized starch, gelatin, sugars (including sucrose, glucose, dextrose, lactose and sorbitol), dextrin, maltodextrin, zein, polyethylene glycol, waxes, natural and synthetic gums such as acacia, guar gum, tragacanth, alginate, sodium alginate, celluloses, including hydroxypropylmethylcellulose, carboxymethylcellulose sodium, bydroxypropylcellulose, hydroxylethylcellulose, ethylcelluloe, methyl cellulose, and veegum, hydrogenated vegetable oil, Type I, magnesium alumninum silicate, and synthetic polymers such as acrylic acid and methacrylic acid copolymers, carbomer, methacrylic acid copolymers, methyl methacrylate copolymers, aminoalkyl methacylate copolymers, polyacrylic acid/polymethacrylic acid, and polyvinylpyrrolidone.
Lubricants

Lubricants are used to facilitate tablet manufacture. Examples of suitable lubricants include, but are not limited to, magnesium stearate, calcium stearate, stearic acid, glyceryl behenate, glyceryl monostearate, glyceryl palmitostearate, hydrogenated castor oil, hydrogenated vegetable oil, type I, sodium benzoate, sodium lauryl sulfate, sodium stearyl fumarate, polyethylene glycol, talc, zinc stearate, and mineral oil and light mineral oil.
Disintegrants

Disintegrants are used to facilitate dosage form disintegration or “breakup” after administration, and generally include, but are not limited to, starch, sodium starch glycolate, sodium carboxymethyl starch, methylcellulose, calcium carboxymethylcellulose, sodium carboxymethylcellulose, hydroxypropyl cellulose, microcrystalline cellulose, colloidal silicon dioxide, croscarmellose sodium, pregelatinized starch, clays, cellulose, powdered cellulose, pregelatinized starch, sodium starch glycolate, sodium aginate, alginic acid, guar gum, magnesium aluminum silicate, polacrilin potassium, and cross linked polymers, such as cross-linked PVP, crospovidone (POLYPLASDONE® XL from GAF Chemical Corp.).

Stabilizers

Stabilizers are used to inhibit or retard drug decomposition reactions which include, by way of example, oxidative reactions. A number of stabilizers may be used. Suitable stabilizers include polysaccharides, such as cellulose and cellulose derivatives, and simple alcohols, such as glycerol; bacteriostatic agents such as phenol, m-cresol and methylparaben; isotonic agents, such as sodium chloride, gycerol, and glucose, lecithins, such as example natural lecithins (e.g. egg yolk lecithin or soya bean lecithin) and synthetic or semisynthetic lecithins (e.g. dimyristoylphosphatidlycholine, dipalmitoylphosphatidylcholine or distearoyl-phosphatidylcholine; phosphatidic acids; phosphatidylethanolamines; phosphatidylserines such as distearoyl-phosphatidylserine, dipalmitoylphosphatidylserine and diarachidoylphospahtidylserine; phosphatidylglycerols; phosphatidylinositols; cardiolipins; sphingomyelins; and synthetic detergents, such as diosetanoylphosphatidyl choline and polyethylene-polypropylene glycol). Other suitable stabilizers include acacia, albumin, alginic acid, bentonite, carboxymethylcellulose calcium, carboxymethylcellulose sodium, cyclodextrins, glyceryl monostearate, hydroxypropyl cellulose, bydroxypropyl methylcellulose, magnesium aluminum silicate, propylene glycol, propylene glycol alginate, sodium alginate, white wax, xanthan gum, and yellow wax. In the preferred embodiment, the agent is insulin and the stabilizer may be a combination of one or more polysaccharides and glycerol, bacteriostatic agents, isotonic agents, lecithins, or synthetic detergents.

Surfactants

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, wafers, films, lozenges, beads, granules, or particles may also contain minor amount of nontoxic auxiliary substances such as dyes, sweeteners, coloring and flavoring agents, pH buffering agents, or preservatives.

Polymers

Blending or copolymerization sufficient to provide a certain amount of hydrophilic character can be useful to improve wettability of the materials. For example, about 5% to about 20% of monomers may be hydrophilic monomers. Hydrophilic polymers such as hydroxypropylcellulose (HPC), hydroxypropylmethylcellulose (HPMC), carboxymethylcellulose (CMC) are commonly used for this purpose. Also suitable are hydrophobic polymers such as polyesters and polyimides. It is known to those skilled in the art that these polymers may be blended with polyanhydrides to achieve compositions with different drug release profiles and mechanical strengths. Preferably, the polymers are bioerodable, with preferred molecular weights ranging from 1000 to 15,000 Da. and most preferably 2000 to 5000 Da.

Film

The composition may be in the form of a film. The film is a clear or opaque, flexible thin material. Typical thicknesses range from 0.01 to 2 mm. The film may have any suitable shape, including round, oval, rectangle, or square. The film may be a monolayer, bilayer or trilayer film. In the preferred embodiment, the film is designed to be suitable for sublingual administration. The monolayer film contains an active agent and one or more excipients. The bilayer film contains one or more excipients, such as a solubilizing agent and/or a metal chelator, in a first layer, and an active agent in the second layer. This configuration allows the active agent to be stored separated from the excipients, and may increase the stability of the active agent, and optionally increases the shelf life of the composition compared to if the excipients and active agent were contained in a single layer. The trilayer film contains three layers of film. Each of the layers may be different, or two of the layers, such as the bottom and top layers, may have substantially the same composition. In one embodiment, the bottom and top layers surround a core layer containing the active agent. The bottom and top layers may contain one or more excipients, such as a solubilizing agent and a metal chelator. Preferably the bottom and top layers have the same composition. Alternatively, the bottom and top layers may contain different excipient(s), or different amounts of the same excipient(s). The core layer typically contains the active agent, optionally with one or more excipients.

In the preferred embodiment, the film is a bilayer film that contains EDTA and citric acid in one layer and insulin in the second layer. Each layer may contain additional excipients, such as glycerin, polyvinyl alcohol, carboxymethyl cellulose, and optionally PEG (such as PEG 400 or PEG 1600). In one embodiment, a third layer can be located between the active agent layer and the layer containing the other ingredients to further protect the active agent from degradative ingredients located in the other layer during storage. Suitable materials for the protective layer include carboxymethylcellulose sodium, carnauba wax, cellulose acetate phthalate, cetyl alcohol, confectioner's sugar, ethylcellulose, gelatin, hydroxyethyl cellulose, hydroxypropyl methylcellulose, liquid glucose, maltodextrin, methylcellulose, microcrystalline wax, polymethacrylates, polyvinyl alcohol, shellac, sucrose, talc, titanium dioxide, and zein.

By altering the composition of the excipients, the film can be designed to dissolve rapidly (less than 30 seconds) or slowly (up to 15 minutes) in order to achieve the desired absorption profile and subsequent effect. The film may dissolve in a time period ranging from 3 to 5 minutes, 5 to 8 minutes, or 8 to 12 minutes. Preferably, the film dissolves in a time period ranging from 15 seconds to 2 minutes.

### Lozenge, Tablet, Capsule, or Wafer

In another embodiment, the composition is in the form of a lozenge, tablet, capsule, or wafer containing the active agent and one or more excipients such as chelators, stabilizing agents, solubilizing agents.

### Lozenge

The lozenge core is composed of a solid gel or a lyophilized wafer, containing an active agent in the core. Optionally, the core also contains a stabilizing agent, optionally with one or more additional excipients. Optionally, the upper and lower surfaces of the lozenge core are coated with a chelator, such as sodium EDTA. Alternatively, the chelator may be mixed with the active agent in the core. In the preferred embodiment, the core contains alginate (preferably calcium stabilized alginate), citric acid, EDTA, and insulin. The lozenge covers a large surface area with a thin layer, and can be made in any convenient shape. Typically it has a round or oval shape. Generally, the lozenge has a diameter and thickness that is approximately the same as the diameter and thickness of a dime. In one embodiment, the lozenge contains glycerine.

### Tablet

In one embodiment, the tablet is a compressed homogenous powder of all of the ingredients. In another embodiment, inactive ingredients, such as the filler and binding agent, and one or more excipients, including the solubilizing agents, are formed into one tablet. The active agent along with filler, binding agent, and other excipients are formed into another tablet. Then the two tablets are placed together and coated to form a single tablet. Optionally, the tablet is coated with an entire coating.

### Wafer

The composition may be in the form of a wafer. The wafer is a flat, solid dosage form. Typical thicknesses range from 0.1 mm to 1.5 cm. Typical diameters range from 0.2 to 5 cm. The wafer may be in any suitable shape, including round, oval, rectangular, or square. The wafer may be a monolayer, bilayer or trilayer. In the preferred embodiment, the wafer is designed to be suitable for sublingual administration. The monolayer wafer contains an active agent and one or more excipients. The bilayer wafer contains one or more excipients, such as a solubilizing agent and/or a metal chelator, in a first layer and an active agent in the second layer. This configuration allows the active agent to be stored separated from the excipients, and may increase the stability of the active agent, and optionally increase the shelf life of the composition compared to if the excipients and active agent were contained in a single layer. The trilayer wafer contains three layers. Each of the layers may be different, or two of the layers, such as the bottom and top layers may have substantially the same composition. In one embodiment, the bottom and top layers surround a core layer containing the active agent. The bottom and top layers may contain one or more excipients, such as a solubilizing agent and a metal chelator. Preferably the bottom and top layers have the same composition. Alternatively, the bottom and top layers may contain different excipient(s), or different amounts of the same excipient(s). The core layer typically contains the active agent, optionally with one or more excipients.

### Capsules

Another suitable dosage form is a capsule. The capsule contains a rapidly dissolving outer shell, which is typically composed of sugars, starches, polymers (and other suitable pharmaceutical materials). The capsule contains powders or granules of agent and excipient. The capsule is designed rapidly release powders or small rapidly dissolving granules into the oral cavity following administration.

### EXAMPLES

The present invention will be further understood by reference to the following non-limiting examples.

The following definitions are used in the examples and figures.
T, time
D, day
Min, minutes
C concentration
Cₘₐₓ, maximum concentration in plasma
t_{Cmax}, time to maximal concentration
tₘₐₓ, time to maximal activity GIRₘₐₓ
t±50% time to half-maximal activity before and after GIRₘₐₓ
GIR, glucose infusion rate
GIRₘₐₓ, glucose infusion rate maximal activity
T_{GIRmax}, time to maximal activity GIRₘₐₓ
T_{GIRmax}, time to maximal activity GIRₘₐₓ
T_{GIR±50%}, time to half-maximal activity before and after GIRₘₐₓ
AUC, area under the curve

The following insulins were used for the studies described herein.
Human insulin, HUMALIN® R, U 100, solution for injection from Eli Lilly and Company, single dose 0.1 JU/kg
Insulin lispro, HUMALOG® R, solution for injection, Eli Lilli and Company, single dose 0.1 IU/kg
Insulin glargine, LANTUS® R, U 100, Sanofi-Aventis, solution for Injection, 0.1 IU/kg

VIAJECT™, solution for injection 25 IU/ml prepared daily from recombinant human insulin, from Biodel Inc. The formulation is:

| Insulin, USP/NF | 0.9 | mg/ml |
|---|---|---|
| Sodium phosphate USP/NF, | 0.7 | mg/ml |
| NaCl, USP/NF | appr. 7.1 | mg/mL |
| Citric Acid, USP/NF | 1.8 | mg/ml |
| Edetate Disodium, EDTA, USP/NF | 1.8 | mg/ml |
| Meta-Cresol | 3.0 | mg/ml |
| HCl/NaOH USP/NF | pH buffer to pH 3.5 to 4.5 (3.95) | |
| Sterile Water, USP/NF | to 10 | ml |

### Example 1

### Comparison of Insulin Size and Absorption With and Without EDTA/Citric Acid

Materials and Methods

VIAJECT™ diluent was added to HUMALOG® and HUMALIN® 1 mg/ml solution in order to achieve a concentration of 0, 1, 2, 3, or 4 mg VIAJECT™ diluent/mL. 0.5 mL of the combined ingredients were added to the top of NANOSEP® microtubes and tubes were spun at 10,000 rpm for 10 minutes in a microcentrifuge (Fisher Scientific). Insulin was assayed before and after the spin, and the percent recovered in the filtrate was determined by dividing the amount of the insulin that filtered through the filter by the initial quantity placed on top.

These were tested to determine apparent permeability as a function of time (minutes) over a period of one hour, and for effect on Tₘₐₓ. Immortalized epithelial cell line cultures were seeded on transwell membranes. When the cells were grown to confluence, at time zero, the fluid in the top chambers of the transwell plates was replaced with 0.5 ml of insulin solution (i.e. solution 1 or solution 2). Two plates with solution 1, two plates with solution 2 and one plate with the control solution (no cells) were tested simultaneously. The lower chamber of each plate contained 1.5 mL of saline solution. At each time point, 100 μL of fluid from the lower chamber was removed and analyzed with Enzyme-Linked Immunosorbent Assay (ELISA). 100 μL of saline was added to the lower chamber to maintain a constant volume of 1.5 mL throughout the study. The amount of insulin removed from the lower chamber at each time point was added to the amount removed in the previous time point(s) to determine the cumulative amount of insulin recovered in the lower chamber.

### RESULTS

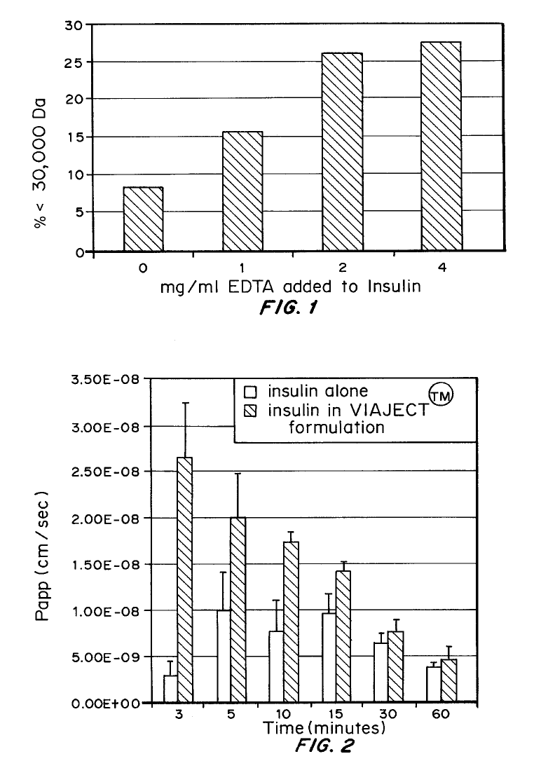

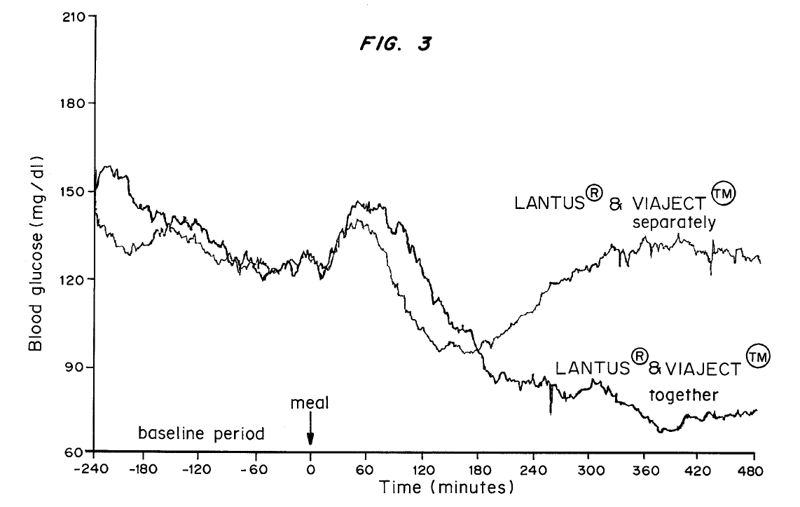

As shown in FIGS. 1 (molecular weight) and 2 (apparent permeability), adding VIAJECT™ (EDTA and citric acid) to insulin results in two populations of molecules, a small size population of insulin molecules and a large size population of insulin molecules. Addition of more EDTA (i.e., a greater amount of VIAJECT™) increases the size of the mean insulin diameter, demonstrating that mean diameter is concentration dependent. The EDTA chelates zinc and charge masks the insulin, which further promotes its absorption across the epithelium, as shown in FIG. 2 as a function of apparent permeability.

As demonstrated by the data for injected insulin shown in Table 1 below, absorption of the VIAJECT™ is so fast that the concentration of insulin in the blood over time resembles the natural initial insulin spike produced by non-diabetic individuals at the beginning of a meal.

TABLE 1
Effect of Chelator/Acid on Tₘₐₓ.

| | ½ T max | T max | −½T max |
|---|---|---|---|
| Type of Insulin | (minutes) | (minutes) | (minutes) |
| Humulin, Regular | 64 | 194 | 325 |
| Human Insulin | | | |
| Humulog, Fast | 52 | 138 | 250 |
| Acting Insulin | | | |
| VIAJECT ™ | 21 | 90 | 210 |
| (Biodel) | | | |

### Example 2

### Co-administration Compared to Administration of Rapid and Long-Lasting Insullin

Materials and Methods

In this study, patients with type 1 diatbetes mellitus were treated with either:
(1) an injection of insulin glargine at a dose equivalent to the subject's usual daily dose of basal insulin and a separate injection of VIAJECT®, or
(2) an injection of insulin glargine at a dose eqivalent to the subject's usual daily dose of basal insulin mixed with VIAJECT™.

### Results

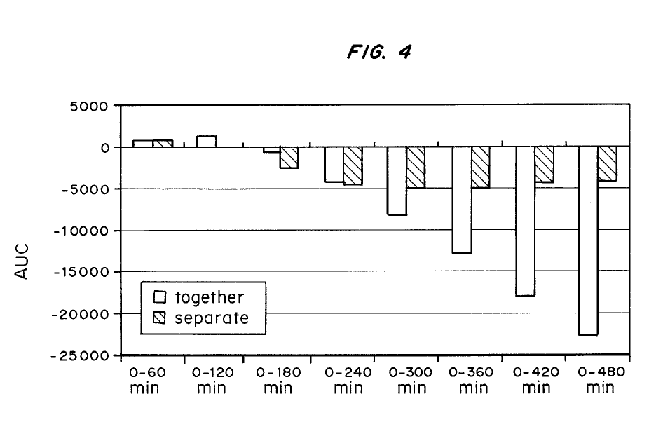

The results are shown in FIGS. 3 and 4. FIG. 3 is a graph of blood glucose (mg/dl) during baseline, at the time of a meal, and following the meal, for the separate injections of LANTUS® and VIAJECT™ as compared to injection of the mixture. FIG. 4 is a graph of the area under the curves at 60, 120, 180, 240, 300, 360, 420, and 480 minutes.

For the first four hours after adminstration, there is no significant difference between LANTUS® and VIAJECT™ mixed together or administered separately, however, after the first four hours are up to at least eight hours, there is a very large and significant difference with the mixture, as compared to the separate injections, of VIAJECT™ and LANTUS® having a much greater effect on lowering blood glucose. The overall significance of this is P<0.004.

TABLE 2
LANTUS t-test

| | p-values |
|---|---|
| AUC0-60 min | 0.962936 |
| AUC0-120 min | 0.195853 |
| AUC0-180 min | 0.264077 |
| Total | 0.000395 |

These results indicate that the chelator in the mixture eftects the biopharmacokinetics.

We claim:

1. A formulation comprising insulin selected from the group consisting of intermediate acting, and long acting insulin with an effective amount of a chelator and an acidifying agent to enhance the rate or amount of uptake by a patient.

2. The formulation of claim 1 further comprising a rapid acting insulin.

3. The formulation of claim 1, wherein the chelator is selected from the group consisting of ethylenediaminetetraacetic acid (EDTA), dimercaprol (BAL), penicillamine, alginic acids, Chlorella, Cilantro, Alpha Lipoic Acid, Dimercaptosuccinic Acid (DMSA), dimercaptopropane sulfonate (DMPS), and oxalic acid.

4. The formulation of claim 3, wherein the chelator is ethylenediaminetetraacetic acid (EDTA).

5. The formulation of claim 1, wherein the agent is a charged compound and wherein the chelator and solubilizing agent are present in effective amounts to mask charges on the agent.

6. The formulation of claim 1 wherein the solubilizing agent is an acid selected from the group consisting of acetic acid, ascorbic acid, citric acid, and hydrochloric acid.

7. The formulation of claim 6, wherein the solubilizing agent is citric acid.

8. The formulation of claim 1, wherein the insulin is natural or recombinant human insulin.

9. The formulation of claim 1 in a solid formulation for administration to a mucosal surface.

10. The formulation of claim 1 in a formulation for administration by injection.

11. A. formulation suitable for pulmonary administration of an insulin in combination with an effective amount of a chelator and an acidifying agent to enhance the rate or amount of uptake by a patient.

12. The formulation of claim 11 wherein the insulin is a natural or recombinant insulin selected from the group consisting of rapid, intermediate and long acting insulins.

13. A method of administering insulin to a patient in need thereof comprising administering the formulation of claim 1 to the patient.

14. The method of claim 13 wherein the formulation is administered to a mucosal surface selected from the group consisting of oral, sublingual, buccal, nasal, rectal, and vaginal.

15. The method of claim 13 wherein the formulation is administered by injection.

16. A method of administering insulin to a patient in need thereof comprising administering the formulation of claim 11 to the pulmonary region of a patient.
